(19) United States
(12) Patent Application Publication
(10) Pub. No.: US 2026/0263687 A1
(43) Pub. Date: Sep. 10, 2026

(54) PUMPING MECHANISM, PUMP AND METHOD OF OPERATING A PUMP

(71) Applicant: Fresenius Vial SAS, Brézins (FR)

(72) Inventor: Damien Archat, Grenoble (FR)

(21) Appl. No.: 19/160,826

(22) PCT Filed: May 22, 2024
(86) PCT No.: PCT/EP2024/064041

§ 371 Date: Aug. 28, 2025

(30) Foreign Application Priority Data
May 24, 2023 (EP) 23315215.6

(51) Int. Cl. A61M 5/142; F04B 43/12

(52) U.S. Cl. CPC A61M 5/14228; F04B 43/1223

(57) ABSTRACT

A pumping mechanism (1), in particular for a pump for administering medical fluid to patient, is provided, wherein the pumping mechanism is configured to act onto a fluid conduit (21) of a pump (2), the pumping mechanism comprising a pumping section (13) movable with respect to said fluid conduit such that a fluid flow through said fluid conduit is generated. The pumping mechanism comprises an eccentric ring (11) for reception of ac shaft of a motor of a pump and a multiplicity of connecting elements (12) each operatively coupled to the pumping section at at least one of a multiplicity of different locations of the pumping section and operatively coupled to the ring in such a way that an eccentric rotational movement of the ring is converted into a translational movement of each of the multiplicity of locations. Furthermore, a corresponding pump (2) and a mechanism of operating a pump is provided.

The invention relates to pumping mechanism, in particular for a pump for administering medical fluid to patient, a pump, in particular for administering medical fluid to a patient, as well as a method of operating a pump.

Pumps use a pumping mechanism to generate a fluid flow through a fluid conduit of the pump in order to deliver a fluid provided to or by the pump to an intended destination. Medical pumps provide medical fluids such as anesthetics, analgesics or medications from a fluid compartment of the pump or from an external fluid compartment to a patient. Different pumping mechanisms for such pumps are known from the prior art. For example, WO 2020/160822 A1 describes a pumping mechanism relying on a multiplicity of rotatable gear elements coupled to a pumping section of the pumping mechanism and coupled to each other for a coordinated rotation of the gear elements for moving the pumping section.

Present pumping mechanisms known in the art are comparatively large, complicated to produce and to maintain. Moreover, most pumping mechanisms exhibit a drift in the pumped volume per cycle to due wear of the pumping mechanism. It is desirable to provide pumping mechanisms for pumps, in particular for medical pumps, which are compact, comparatively easy and cost-effective to produce and durable.

It is an object of the instant invention to provide a pumping mechanism which is compact, durable and immune to wear and tear as well as easy and cost-effective in production.

This object is achieved by means of a pumping mechanism comprising the features of claim 1.

Accordingly, the pumping mechanism is configured to act onto a fluid conduit of a pump. The pumping mechanism comprises a pumping section which is movable with respect to said fluid conduit such that a fluid flow through said fluid conduit is generated through movement of the pumping section. The invention is characterized in that the pumping mechanism comprises an eccentric ring for reception of a shaft of a motor of a pump. Further, the pumping mechanism comprises a multiplicity of connecting elements, each operatively coupled to the pumping section at at least one of a multiplicity of different locations of the pumping section and further operatively coupled to the ring in such a way that an eccentric rotational movement of the ring is converted into a translational movement of each of the multiplicity of locations of the pumping section.

Herewith, a pumping mechanism is provided which is compact, in particular flat, can be produced cost-effectively and comparatively easily. Moreover, the pumping mechanism according to the invention is durable, hence providing an extended lifetime as well as a reduced drift in the pumped volume per cycle.

The pumping mechanism is, for example, a pumping mechanism of a pump for administering medical fluid to a patient, e.g. an infusion pump.

According to one aspect, each connecting element is directly coupled to the ring via a pivot, in particular a flexural pivot. The pivot allows for a flexible connection between the connecting element and the ring. The flexural pivot additionally provides a durable, cost-effective pivot. In one example, the flexural pivot is a small-length flexural pivot, e.g. a bendable blade.

According to one aspect, at each location of the multiplicity of locations, a pivot, in particular a flexural pivot, is provided for flexibly coupling the respective connecting element to the pumping section. In one example, the flexural pivot is a small-length flexural pivot or a cross-axis flexural pivot. The small-length flexular pivot is e.g. a single bendable blade. The cross-axis flexural pivot comprises e.g. crossed bendable blades. By flexibly coupling the connecting elements to the pumping section at a multiplicity of locations, the connecting elements can exert a force on the pumping section at the respective location and hence, generate a fluid flow in a fluid conduit of a pump.

According to one aspect, the connecting elements face radially outward from the ring, preferably at equal angular distances on the ring. The connecting elements facing radially outward from the ring allow an eccentric rotational movement of the ring, e.g. through a driving force exerted by the motor via a shaft to the ring, being translated into a translational movement of the pumping section at the multiplicity of locations of the pumping section. An arrangement at equal angular distances allows for a defined phase shift of the motion of the pumping section at the respective locations.

According to one aspect, the multiplicity of connecting elements comprises a first vertical connecting element arranged on the side of the ring facing the pumping section, wherein the first vertical connecting element is directly coupled to the pumping section at a first location of the multiplicity of locations via the pivot, in particular the flexural pivot, at that location. Hence, an eccentric rotational movement of the ring is converted into a translational movement of the corresponding location on the pumping section. The flexural pivot provides the necessary flexibility of the connection at the location. In one variant, the first vertical connecting element is coupled to the pumping section at a center of the pumping section, i.e. in the middle between a first and a second end of the pumping section. The first and second end are defined as the areas of the pumping section in which a fluid enters and leaves the pumping mechanism, respectively, when the pumping mechanism is in operation. That means, the first location is at a center of the pumping section, also referred to as center location.

According to one aspect, the first vertical connecting element is additionally coupled to a double-bell-crank mechanism for redirecting a downwards movement of the first vertical connecting element into an upwards movement of a second and third location of the pumping section to which the first vertical connecting element is additionally coupled to. The double-bell-crank mechanism comprises a first bell-crank via which the first vertical connecting element is coupled to a second location of the pumping section via a first linking element. The double-bell-crank mechanism further comprises a second bell-crank via which the first vertical connecting element is coupled to a third location of the pumping section via a second linking element. The first and second bell-crank of the double-bell-crank mechanism are coupled to the first vertical connecting element via a pivot. The first vertical connecting element is, e.g. a rod with thinned portion along its length, wherein the thinned portion is part of a pivot comprising two crossed bendable blades, wherein the first bendable blade, formed by the thinned portion, extends in the direction of the first vertical connecting element and the second bendable blade connects the first bell-crank and the second bell-crank of the double-bell-crank mechanism in a direction perpendicular to the first vertical connecting element. The first and second bell-crank is coupled to the first and second linking element e.g. via a pivot, respectively.

According to an alternative aspect, the multiplicity of connecting elements comprises a second vertical connecting element arranged on the side of the ring facing away from the pumping section, wherein the second vertical connecting element is for example coupled to the pumping section via a sleeper-bell-crank-mechanism for redirecting a downwards movement of the second vertical connecting element, i.e. a movement away from the pumping section, into an upwards movement of a corresponding at least one location of the pumping section to which the second vertical connecting element is coupled to. Here and in the following, “upwards” and “downwards” refer to movements perpendicular to the plane defined by the pumping section at rest, wherein “upwards” refers to a movement from the ring-side towards the pumping section and “downwards” to a movement from the ring-side away from the pumping section. In one example, the first vertical connecting element and the second vertical connecting element are coupled to the ring at opposite sides of the ring, i.e. the angle between both connecting elements on the ring is 180°.

According to an aspect, the sleeper-bell-crank mechanism comprises a first bell-crank for coupling the second vertical connecting element to a sleeper and a first and a second side bell-crank for coupling the sleeper via a first and a second intermediate linking element, in particular a first and a second rod, to the pumping section at a second location and at a third location of the multiplicity of locations via the pivot, in particular the flexural pivot, at the second location and via the pivot, in particular the flexural pivot, at the third location, respectively. In this way, an upward movement of the second vertical connecting element is translated in a downward movement of the pumping section at the second and third location. The second and third location are, for example, located at the first and the second end of the pumping section thus defining a first end location and a second end location. In this way, a movement of the pumping section is provided, wherein the first and the second end of the pumping section are moved simultaneously in the same direction.

According to one aspect, the multiplicity of connecting elements comprises a second vertical connecting element arranged on the side of the ring facing away from the pumping section, wherein the second vertical connecting element is for example coupled to the pumping section via a double-bell-crank mechanism for redirecting a downwards movement of the second vertical connecting element into an upwards movement of a corresponding at least one location of the pumping section to which the second vertical connecting element is coupled to. In one example, the first vertical connecting element and the second vertical connecting element are coupled to the ring at opposite sides of the ring, i.e. the angle between both connecting elements on the ring is 180°. Through this setup, the volume requirement of the pumping mechanism is reduced.

According to one aspect, the corresponding double-bell-crank mechanism comprises a first bell-crank for coupling the second vertical connecting element to the pumping section at a second location via a first intermediate linking element, in particular a first rod. The double-bell-crank mechanism further comprises a second bell-crank for coupling the second vertical connecting element to the pumping section at a third location via a second intermediate linking element, in particular a second rod. The first and second intermediate linking element are coupled to the second and third location via the pivot, in particular the flexural pivot, provided at the second and third location, respectively. In this way, an upward movement of the second vertical connecting element is translated in a downward movement of the pumping section at the second and third location. The second and third location are, for example, located at the first and the second end of the pumping section thus defining a first end location and a second end location. In this way, a movement of the pumping section is provided, wherein the first and the second end of the pumping section are moved simultaneously in the same direction.

According to a further aspect, the first and second vertical connecting elements are arranged along a vertical axis, wherein the vertical axis extends perpendicular to a plane defined by the pumping section at rest and crosses the center of the ring. Thus, a rotational movement of the ring is translated into a translational movement of the locations of the pumping section to which the first and second vertical connecting elements are coupled to, wherein the translational movement is approximately perpendicular to the plane defined by the pumping section at rest. As pointed out above, a movement along this vertical axis will also be referred to as an upwards or downwards movement, whereas upwards refers to a movement of the pumping section along the vertical axis in the direction on the ring's side towards to the pumping section, whereas downwards refers to a movement of the pumping section along the vertical axis on the ring's side away from the pumping section. Hence, the first vertical connecting element provides an up-and downwards movement of the pumping section at the first location and the second vertical connecting element provides an up-and downwards movement of the pumping section at the second and third locations. In a variant, where the first vertical connecting element and the second vertical connecting element are spaced 180° apart on the ring, and the first location is a center location of the pumping section, the movement of the center location has a phase shift of 180° with respect to the movement of the third and second locations, in particular the first and second end locations. That means, when the center location moves upwards, the first and second end locations move downwards and vice versa. Thus, a fluid flow through a fluid conduit in the direction from the first to the second end of the pumping section can be generated.

In a further aspect, the multiplicity of connecting elements comprises at least one intermediate connecting element, wherein the intermediate connecting element is coupled to the pumping section via a bell-crank, wherein the bell-crank is coupled to the pumping section at one location of the multiplicity of locations via a linking element and via the pivot, in particular the flexural pivot, at that location. Thus, an additional location is provided on the pumping section, at which a pumping force can be exerted on the pumping section. In particular, the bell-crank compensates for movements of the intermediate connecting element parallel to the plane defined by the pumping section at rest, whereas movements perpendicular to that plane are converted into up and down movements of the pumping section at the respective location of the pumping section to which the intermediate connecting element is coupled to. In one example, the location to which the intermediate connecting element is connected is intermediate between the first and second end location of the pumping section, in particular between the first end location and the center location or between the second end location and the center location. Preferably, the location to which the at least one intermediate connecting element is coupled to is determined in dependence of the angular distance between the first vertical connecting element and the intermediate connecting element on the ring and/or the angle between the second vertical connecting element and the intermediate connecting element on the ring. E.g. if the intermediate connecting element is coupled to the ring at an angular distance on the ring halfway between the first vertical connecting element and the second vertical connecting element, the location on the pumping section to which the intermediate connecting element is coupled is halfway between the first location and the second or the third location. In this way, a wavelike pumping movement is provided by the pumping section and a fluid flow can be generated in a fluid conduit.

According to one aspect, the multiplicity of connecting elements consists of four connecting elements facing radially outward from the ring. In an alternative embodiment the multiplicity of connecting elements consists of eight connecting elements facing radially outward from the ring. With four connecting elements, a movement of the pumping section for generating a fluid flow in a fluid conduit can be provided in a simple, yet efficient way. In one example, the four connecting elements comprise a first and a second vertical connecting element as well as a first and a second intermediate connecting element, wherein the first and the second intermediate connecting elements are coupled to the ring spaced at an angle of 90° and 270° from the first vertical connecting element, respectively. In particular, the second vertical connecting element is coupled to the first end location and the second end location of the pumping section. The first vertical connecting element is, for example, coupled to the center location. In one example, the first and second intermediate connecting elements are coupled to the pumping section at a location halfway between the first end location and the center location and at a location halfway between the center location and the second end location. In this way, a wavelike movement of the pumping section can be provided through a rotational movement of the ring. Thus, a fluid flow through a fluid conduit can be easily and efficiently generated. In one example, four additional intermediate connecting elements are provided at an angular distance on the ring of 45°, increasing the number of locations at which an up-and downwards force is exerted on the pumping section contributing to a pumping movement of the pumping section.

According to one aspect, the multiplicity of connecting elements consists of three connecting elements facing radially outward from the ring. In one example, the three connecting elements comprise a first vertical connecting element and a first and a second intermediate connecting element, wherein the first and second intermediate connecting elements are coupled to the ring spaced at an angle of 90° and 270° from the first vertical connecting element, respectively. In particular, the first vertical connecting element is directly coupled to the first location and furthermore coupled to a second and a third location via the double-bell-crank mechanism. The first location is, e.g. a center location of the pumping section and the second and third location a first and a second end location of the pumping section. The first and second intermediate connecting elements are, for example, coupled to locations between the center location and the respective end location via a first and a second bell-crank. The first and second bell-crank coupling the first and second intermediate connecting elements to the pumping section overlap the first and second bell-crank of the double-bell-crank mechanism, for example.

In one aspect, the pumping mechanism comprises at least one fixation element for connecting the pumping mechanism to a housing of a pump. For example, the first and second bell-crank of the sleeper-bell-crank mechanism are each coupled to a fixation element for connecting the pumping mechanism to a housing of a pump. Alternatively, the first and second bell-crank of the double-bell-crank mechanism are each coupled to a fixation element for connecting the pumping mechanism to a housing of a pump. In particular, the fixation element is provided inside the first and/or second bell-crank of the double-bell-crank mechanism. A pivot is, for example, provided for coupling the first and second bell-crank to the respective fixation element. This reduces the volume of the pumping mechanism considerably.

According to one aspect, each of the multiplicity of connecting elements is a rod. In this way, movement of the pumping mechanism relative to a housing of a pump is prevented. Rods provide connecting elements which are easy to produce and to maintain.

According to an aspect, the pumping mechanism is formed as a single piece. That means, the pumping mechanism is monolithic, simplifying assembly and production, as well as mounting and replacement in a pump. For example, the pumping mechanism is formed as a single plastic piece, e.g. through plastic injection or through 3D-printing. In particular, the pumping mechanism comprising three connecting elements with overlapping bell-cranks is produced through 3D-printing. 3D-printing provides an efficient way of producing pumping mechanisms of comparatively small volume, in particular by allowing to arrange parts in an overlapping manner. 3D-printing comprises the built-up of a three-dimensional structure, here a pumping mechanism, layer by layer e.g. from a liquid plastic material. Furthermore, no grease is necessary to reduce friction between different parts as all components are formed into a single piece. In particular, the use of flexural pivots makes grease superfluous. In this way, hygiene is improved.

According to a further aspect, the pumping section comprises a multiplicity of pumping elements, wherein neighbouring pumping elements of the multiplicity of pumping elements are pivotally coupled to each other at each of the locations. In this way, pinching zones are provided which, upon an upwards movement of the respective pumping element, reduce (pinch) the diameter of a fluid conduit in the area of the pinching zone. Hence, a fluid flow can be generated in a fluid conduit.

It is a further object of the invention to provide a pump with a compact, durable and cost-effective pumping mechanism.

This object is achieved by means of a pump comprising the features of claim 14. Accordingly, the pump comprises a fluid conduit and a motor. The pump is characterized by a pumping mechanism comprising the features of the claims 1 to 13. In particular, the pump comprises a ring which is eccentrically coupled to a shaft of the motor of the pump. Hence, the motor of the pump provides an eccentric rotational movement which is translated into a linear peristaltic movement of the pumping section by the pumping mechanism.

A further object of the present invention is to provide a method of operating a pump which allows for a compact, durable and cost-effective pump.

This object is achieved by means of a method according to claim 15. Accordingly, the method comprises the steps of acting, through a pumping mechanism of a pump, onto a fluid conduit of a pump, wherein a pumping section of the pumping mechanism generates a fluid flow through said fluid conduit through movement of the pumping section.

The method is characterized in that the method comprises the step of moving the pumping section by a translational movement of a multiplicity of locations of the pumping section by a multiplicity of connecting elements which are operatively coupled to the pumping section at at least one of the multiplicity of different locations of the pumping section, wherein each of the multiplicity of connecting elements is coupled to an eccentric ring of the pumping mechanism for reception of a shaft of a motor, through conversion of an eccentric rotational movement of the ring into the translational movement of the multiplicity of locations.

The pumping mechanism is, for example, a pumping mechanism comprising the features of the claims 1 to 13. The pump is, for example, a pump comprising the features of claim 14. The pump is, for example, a pump for administering a medical fluid to a patient, e.g. an infusion pump.

The idea underlying the invention shall subsequently be described in more detail with respect to the embodiment shown in the drawings. Herein:

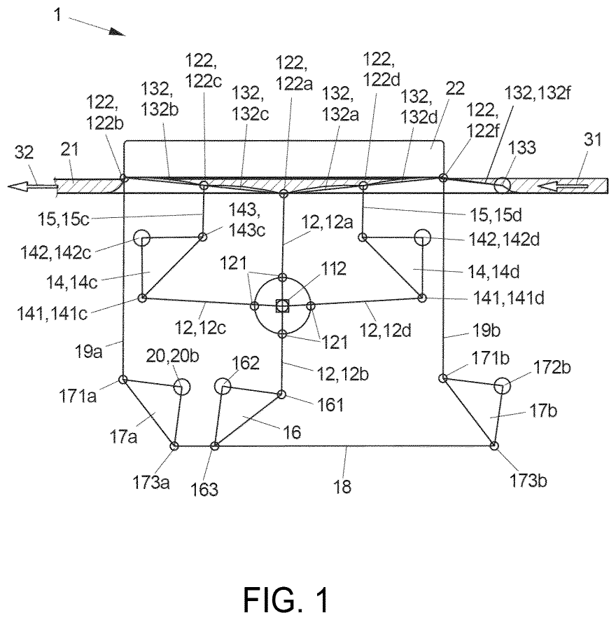

FIG. 1 shows a schematic illustration of a pumping mechanism according to a first embodiment of the present invention;

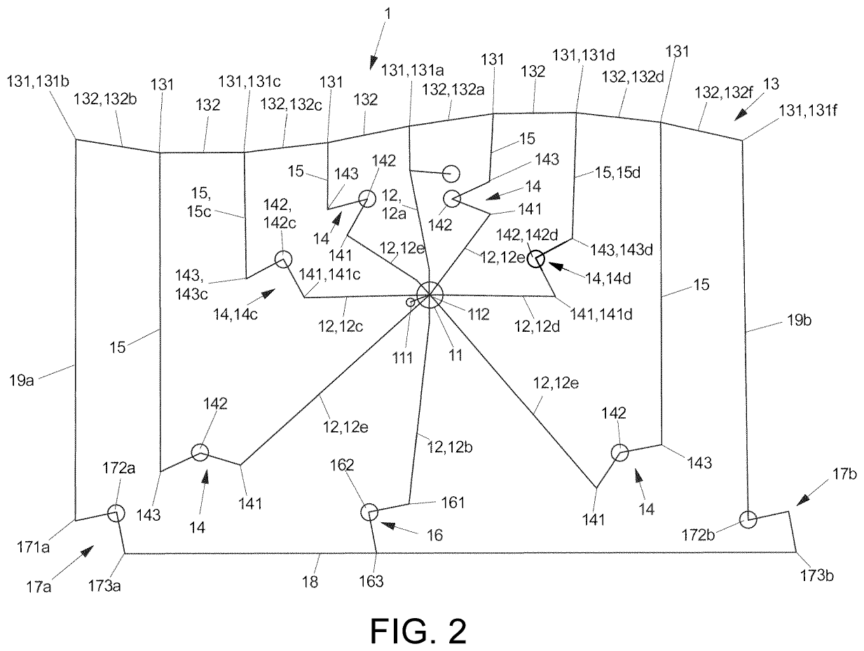

FIG. 2 shows a schematic illustration of a pumping mechanism according to a second embodiment of the present invention;

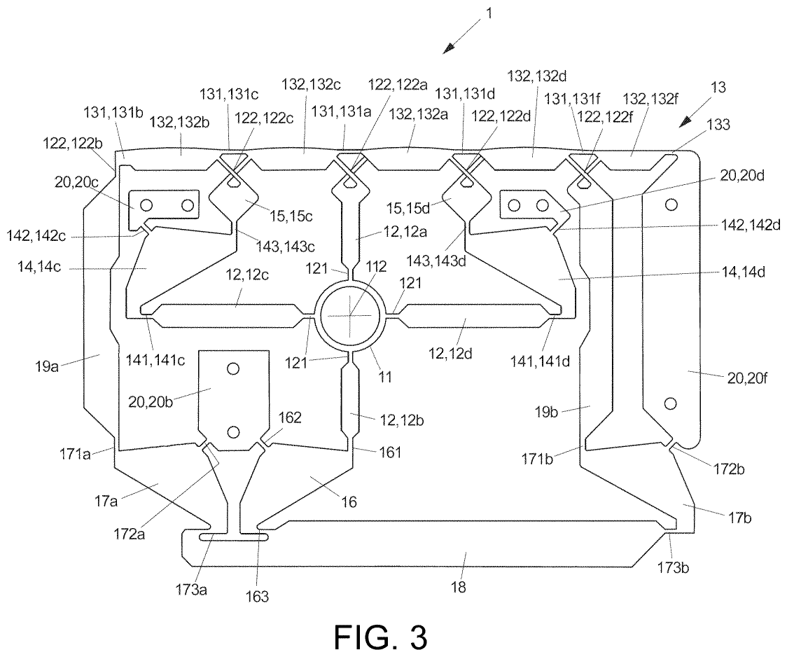

FIG. 3 shows a schematic illustration of a pumping mechanism according to a third embodiment of the present invention;

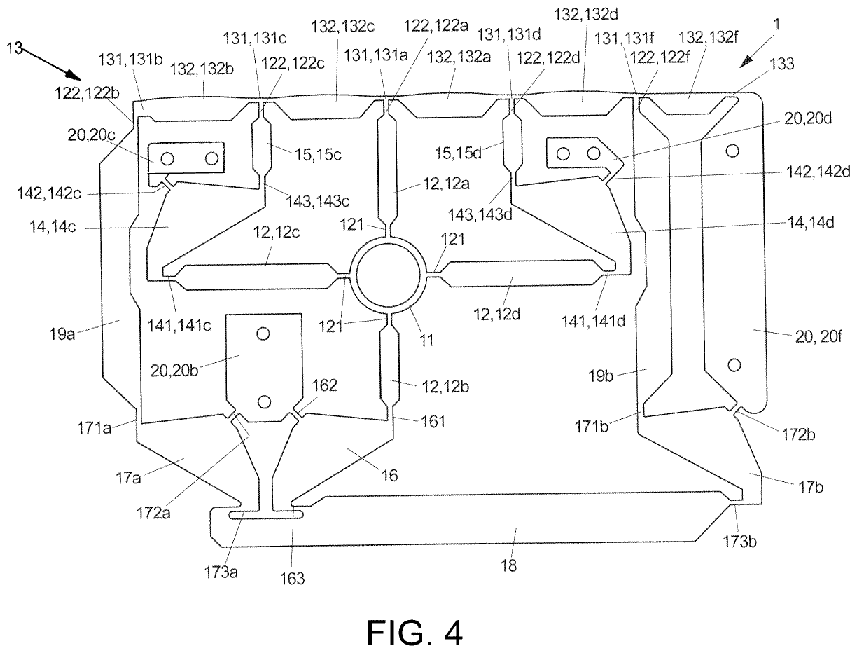

FIG. 4 shows a schematic illustration of a pumping mechanism according to a fourth embodiment of the present invention;

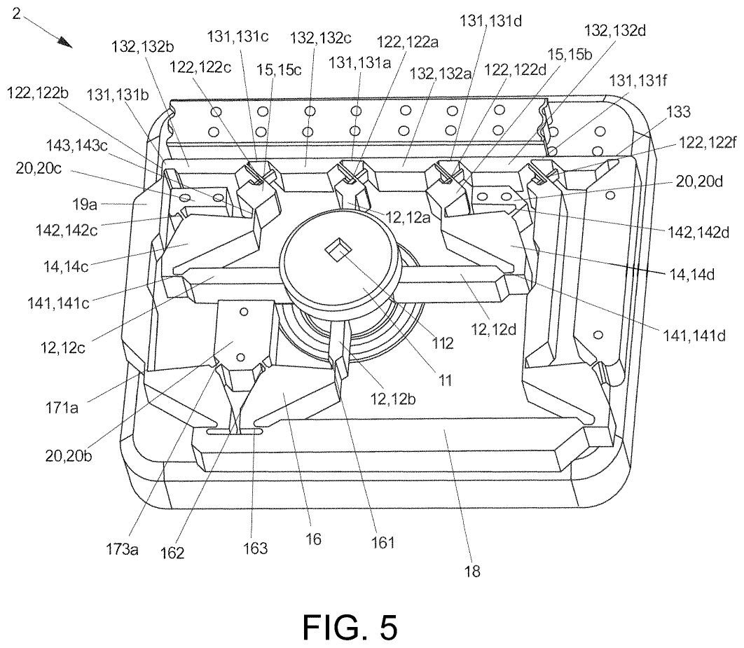

FIG. 5 shows a sectional view of a part of a pump according to the present invention;

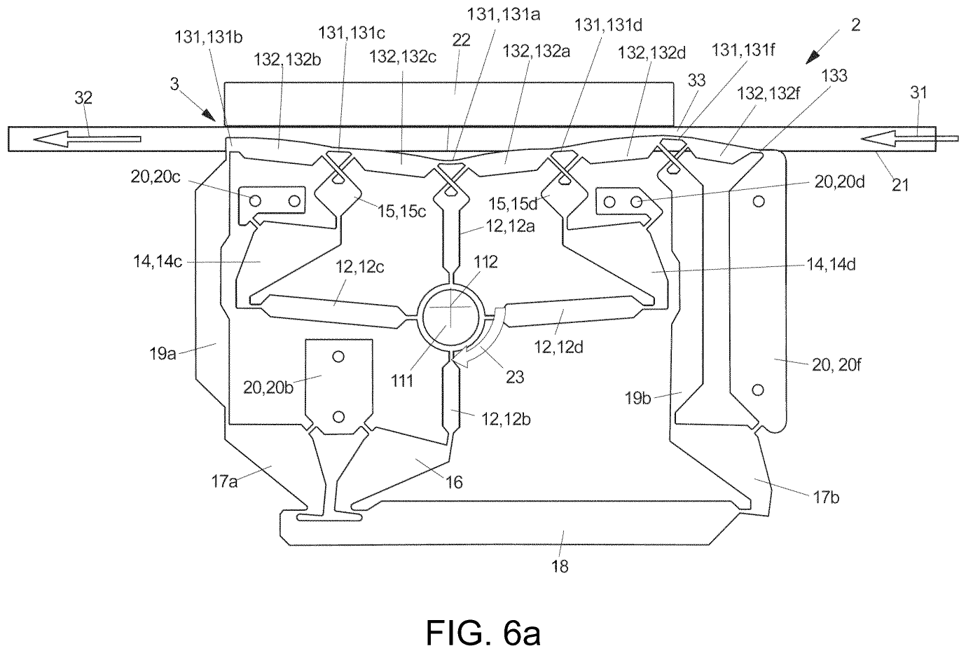

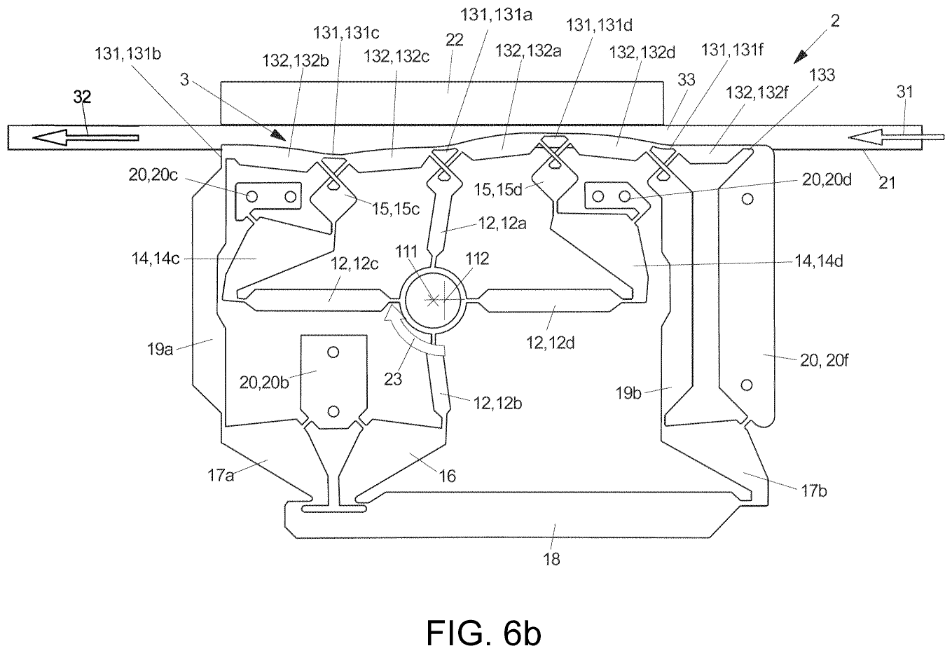

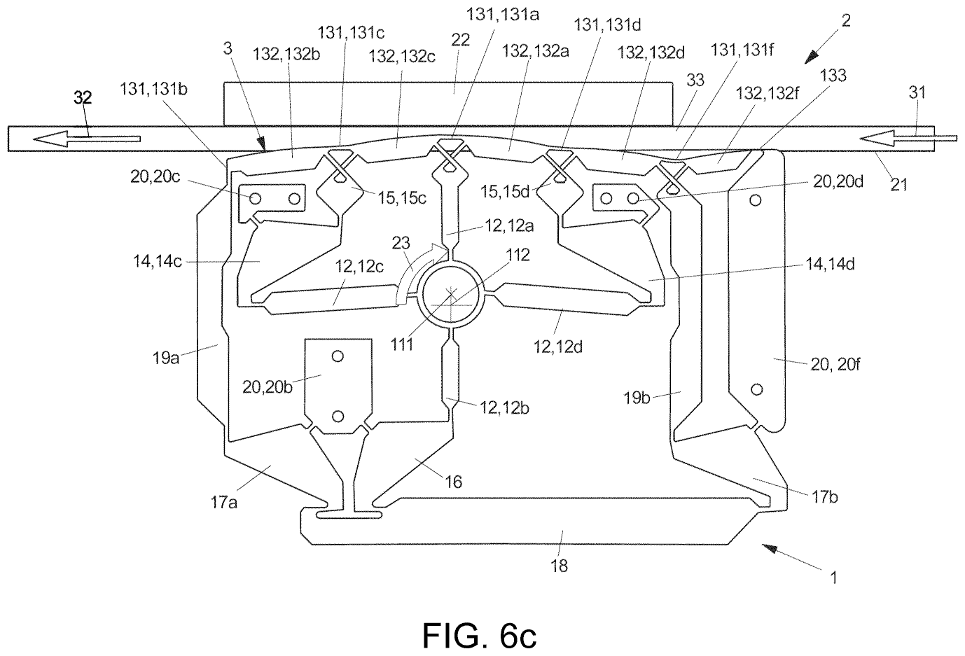

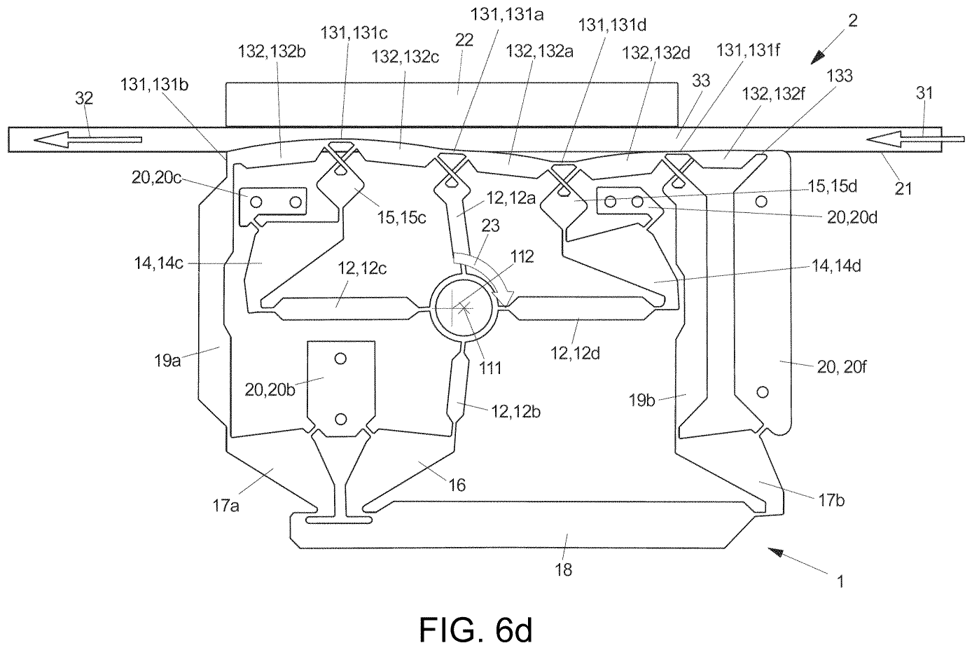

FIG. 6a-FIG. 6d illustrate a method according to an embodiment of the present invention.

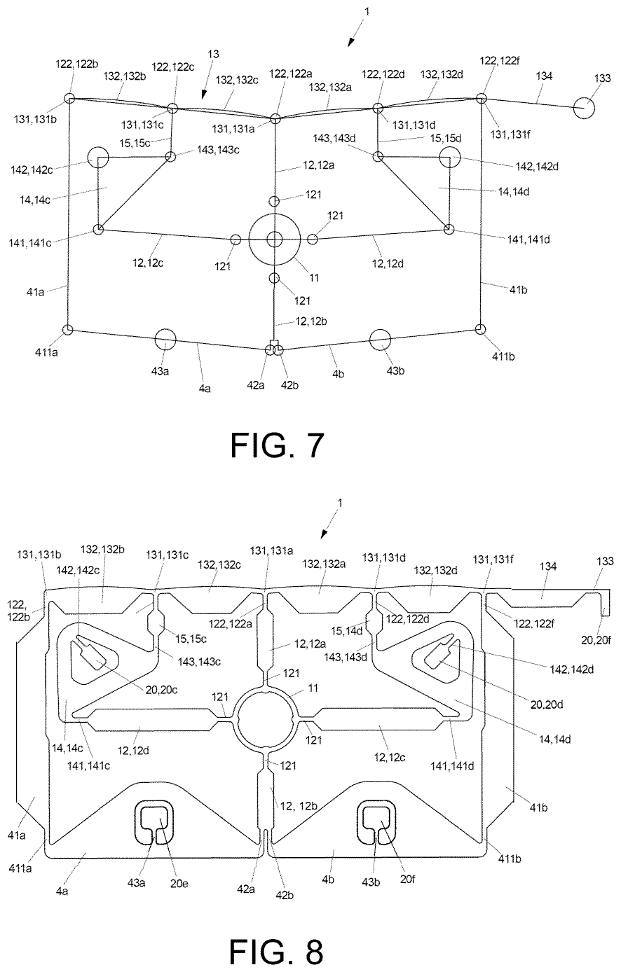

FIG. 7 shows a schematic illustration of a pumping mechanism according to a fifth embodiment of the present invention;

FIG. 8 shows a schematic illustration of a pumping mechanism according to the fifth embodiment of the present invention; and

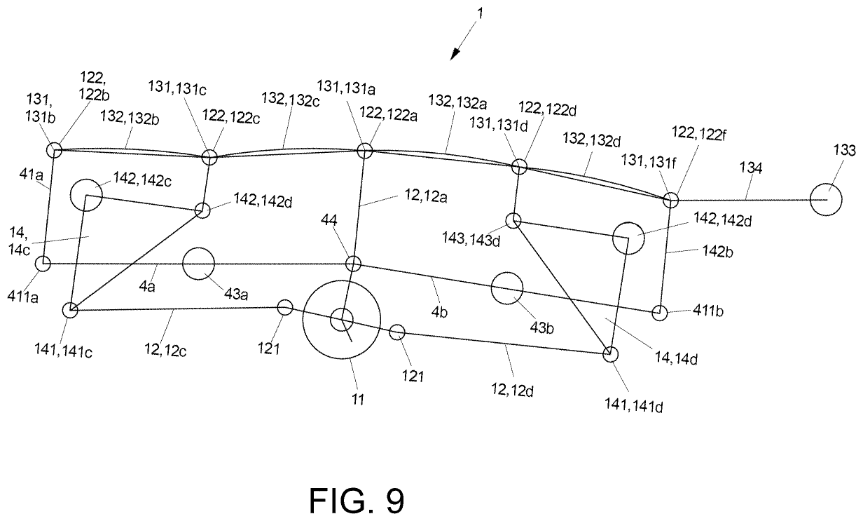

FIG. 9 shows a schematic illustration of a pumping mechanism according to a sixth embodiment of the present invention.

FIG. 1 shows a pumping mechanism 1 according to an embodiment of the present invention. The pumping mechanism 1 is configured to act onto a fluid conduit 21 of a pump 2 (see FIG. 5). The pumping mechanism 1 comprises a pumping section 13 which is movable with respect to said fluid conduit 21. Through this movement of the pumping section 13 a fluid flow is generated in the fluid conduit 21. An upstream incoming fluid 31 is pumped by the pumping mechanism 1 from a first end of the fluid conduit 21, producing a downstream outcoming fluid 32 at a second end of the fluid conduit 21. For example, the pumping mechanism 1 is a pumping mechanism 1 for a pump 2 for administering medical fluid to a patient and the fluid to be pumped is a medical fluid such as medication, an anaesthetic or an analgesic.

The pumping mechanism 1 comprises an eccentric ring 11 for reception of a shaft of a motor of a pump 2. The ring 11 comprises, for example, a receptacle 111 for a motor shaft (see e.g. FIG. 2) and an eccentric 112 applied to that motor shaft. The motor can thus drive, via the shaft, an eccentric movement of the ring 11. The pumping mechanism 11 further comprises a multiplicity of connecting elements 12. In the present example, the pumping mechanism 1 comprises four connecting elements 12. The connecting elements 12 are each coupled to the pumping section 13 at at least one of a multiplicity of locations 131. The connecting elements 12 are also operatively coupled to the ring 11 in such a way that a rotational movement of the ring 11, in particular the movement generated by the driving force of a motor of a pump 2, is converted into a translational movement of each of the multiplicity of locations 131 for moving the pumping section 13. In particular, the pumping section 13 is subject to a translational movement driven by the connecting elements 12 at the respective locations 131 at which the connecting elements 12 are operatively coupled to the pumping section 13. In the present example four connecting elements 12 are coupled to the pumping section 13 at five locations 131. The pumping section 13 comprises a multiplicity of pumping elements 132. Neighbouring pumping elements 132 are pivotally coupled to each other at each of the locations 131. Hence, in the present example with five locations 131, four pumping elements 132 are provided. Each connecting element 12 is coupled to the ring 11 via a pivot 121, in particular a flexural pivot. The coupling of the connecting elements 12 to the pumping section 13 at the respective location 131 is also provided by a pivot 122, for example a flexural pivot, in particular a small-length flexural pivot or a cross-axis flexural pivot. The connecting elements 12 face radially outward from the ring 11. Preferably, they are coupled to the ring 11 at equal angular distances on the ring. In the present example, the angular distance between the connecting elements 12 is 90°. In a resting position, i.e. in the absence of an external force exerted by a pump motor, the connecting elements 12 face radially outward from the ring 11 at angles of 0°, 90°, 180° and 270°, respectively, wherein an angle of 0° is defined by a vertical axis perpendicular to the plane spanned by the pumping section 13. The multiplicity of connecting elements 12 comprises a first vertical connecting element 12a. This first vertical connecting element 12a is arranged on the side of the ring 11 which faces the pumping section 13. The first vertical connecting element 12a is directly coupled to the pumping section 13 at a first location 131a via the pivot 122a at the first location 131a. In the depicted example, the first vertical connecting element 12a is arranged along a vertical axis, wherein the vertical axis extends perpendicular to a plane defined by the pumping section 13 when at rest and crosses the center of the ring 11. In one example, the first location 131a is at the center of the pumping section 13. That means, the first location 131a in this example is located halfway between a first end and a second end of the pumping section 13. The multiplicity of connecting elements 12 also comprises a second vertical connecting element 12b. The second vertical connecting elements 12b is arranged on the side of the ring 11 facing away from the pumping section 13. It is coupled to the pumping section 13 via a sleeper-bell-crank mechanism for redirecting a movement of the second vertical connecting element 12b towards the pumping section 13. In particular, the second vertical element 12b is also arranged on the vertical axis which extends perpendicular to the plane defined by the pumping section 13 when at rest and crosses the center of the ring 11. The sleeper-bell-crank mechanism comprises a sleeper 18 and a first bell crank 16 for coupling the second vertical connecting element 12b to the sleeper 18. The sleeper-bell-crank mechanism further comprises a first and a second side bell-crank 17a, 17b for coupling the sleeper 18 via a first and a second linking element 19a, 19b to the pumping section 13 at a second and third location 131b, 131f. The first and second linking element 19a, 19b are coupled to the second and third location 131b, 131f via a pivot 122b, 122f at the respective location 131b, 131f. The second and third location 131b, 131f are located, for example, at the first end and at the second end of the pumping section 13, respectively. Hence, the first linking element 19a is coupled to a first end location and the second linking element 19b is coupled to a second end location. The multiplicity of connecting elements 12 in the present example comprises a first and a second intermediate connecting element 12c, 12d. Each intermediate connecting element 12c, 12d is coupled to the pumping section 13 at a respective location 131c, 131d via a bell-crank 14c, 14d, wherein the respective bell-crank 14c, 14d is coupled to the pumping section 13 at location 131c, 131d via a pivot 122c, 122d, respectively. A linking element 15c, 15d is provided, in order to couple the bell-crank 14c, 14d to the location 131c, 131d via the pivot 122c, 122d, respectively. Fixation elements 20 are provided for reversibly or irreversibly connecting the pumping mechanism 1 to a housing 22 of a pump 2. In the present example, the bell-cranks 14c, 14d, 16, 17a, 17b are coupled to fixation elements 20c, 20d, 20b, 20b, 20f, respectively. The coupling is achieved in particular via a pivot 142c, 142d, 162, 172a, 172b, respectively. Further, a holding link 134 is pivotally coupled to pivot 133 coupling the holding link 134 to a fixation element. In one example, the connecting elements 12 are rods, i.e. the first and second vertical connecting elements 12a, 12b as well as the first and second intermediate connecting elements 12c, 12d are rods. In this way, through eccentric rotation of the ring 11, the pumping section 13 is moved at the locations 131a, 131b, 131c, 131d and 131f. The movement is directed along the vertical axis perpendicular to the plane defined by the pumping section 13 when at rest. This movement is described as an up-or downwards movement of the respective locations 131a, 131b, 131c, 131d and 131f. Due to the angular spacing of the connecting elements 12a, 12b, 12c, 12d, the up-and downwards movements at the locations 131a, 131b, 131c, 131d, 131f are phase shifted with respect to each other. The movement at neighbouring locations 131a, 131b, 131c, 131d, 131f is phase shifted by 90°. Thus, a wavelike movement of the pumping section 13 results, generating a fluid flow in a fluid conduit 21 of a pump 2.

FIG. 2 illustrates a pumping mechanism 1 according to another embodiment of the present invention. The embodiment depicted in FIG. 2 differs from the embodiment depicted in FIG. 1 with regard to the number of connecting elements 12. The presently illustrated embodiment has, in addition to the connecting elements 12 of the embodiment of FIG. 1, four additional intermediate connecting elements 12e. All connecting elements 12 are coupled to the ring 11 at equal angular distances. That means that the coupling points of the connecting elements 12 are spaced apart by 45° instead of 90° as in the example of FIG. 1. Thus, additional locations 131e are provided on the pumping section 13 at which the pumping section 13 is moved up-and downwards through an eccentric rotational movement of the ring 11. The phase shift of the movements of neighbouring locations 131 is 45° in this embodiment. As can be inferred from FIG. 2, the ring 11 comprises, for example, a receptacle 111 for a motor shaft and an eccentric 112 applied to that motor shaft.

FIG. 3 shows a pumping mechanism 1 in a further embodiment of the present invention. The embodiment of FIG. 3 is a further specification of the embodiment of FIG. 1. Hence, the presently described embodiment has at least the features of the embodiment described with regard to FIG. 1. In the depicted embodiment, all pivots are carried out as flexural pivots. Flexural pivots have the advantage that they are easy and comparatively cost-effective to produce. Moreover, no grease needs to be provided and hence, hygiene is improved. For example, the connecting elements 12 are coupled to the pumping section 13 at respective locations 131 through flexural pivots 122. In particular, the pivot 122a which couples the first vertical connecting element 12a directly to the first location 131a at the center of the pumping section 13 is a cross-axis flexural pivot. The pivot 122b coupling the second vertical connecting element 12b, via the sleeper-bell-crank mechanism, to the pumping section 13 at a second and third location 131b, 131f is a flexural pivot. In the depicted example, the pivot 122b coupling the first linking element 19a to the second location 131b is a small-length flexural pivot. The pivot 122f coupling the second linking element 19b of the sleeper-bell-crank mechanism to the third location 131f is a cross-axis flexural pivot. The pivot 133 coupling the holding link 134 to the fixation element 20f is a small-length flexural pivot. The pivots 122c, 122d coupling the first and the second intermediate connecting elements 12c, 12d to the respective location 131c, 131d of the pumping section 13 is a flexural pivot, in particular a cross-axis flexural pivot. Hence, whereas the inner pumping elements 132c, 132a, 132d are coupled directly or indirectly to the connecting elements 12c, 12a and 12d, respectively, via cross-axis flexural pivots (122c, 122a, 122d in this example), the outer pumping element 132b is coupled to the first linking element 19a and the holding link 134 is coupled to the fixation element 20f via small-length flexural pivots (122b, 133 in this example), respectively. The pivots 121 coupling the connecting elements 12a, 12b, 12c, 12d are coupled to the ring 11 via small-length flexural pivots. The pivots 141c, 141d coupling the first and the second intermediate connecting element 14c, 14d to the first and second bell-crank 14c, 14d, respectively, are embodied as small-length flexural pivots. Likewise, the pivot 161 coupling the second vertical connecting element 12b to the first bell-crank 16 of the sleeper-bell-crank-mechanism is a small-length flexural pivot. The pivots 142c, 142d, 162, 172a, 172b coupling the bell-cranks 14c, 14d, 16 as well as the first and second side bell-cranks 17a, 17b to the respective fixation elements 20c, 20d, 20b, 20b, 20f, respectively, are small-length flexural pivots. The pivots 163, 173a, 173b coupling the first bell-crank 16 of the sleeper-bell-crank mechanism as well as the first and second side bell-cranks 17a, 17b to the sleeper 18 are small-length flexural pivots. Likewise, the pivots 171a, 171b coupling the first and second rods 19a, 19b to the first and second side bell-crank 17a, 17b, respectively, are small-length flexural pivots. The pivots 143c, 143d coupling the linking elements 15c, 15d to the first and second bell-crank 14c, 14d are small-length flexural pivots. In one example, the entire pumping mechanism 1 is formed as a single piece, for example through plastic injection.

FIG. 4 provides an alternative embodiment of the pumping mechanism 1. The pumping mechanism 1 of this embodiment differs from the embodiment depicted in FIG. 3 in that instead of cross-axis flexural pivots, small-length flexural pivots are used for all pivots. In particular, the pivots 122a, 122b, 122c, 122d, 122f coupling the connecting elements 12 directly or indirectly to the pumping section 13 are all provided as small-length flexural pivots. This further simplifies the production and reduces cost.

FIG. 5 illustrates a part of a pump 2 with a pumping mechanism 1 according to the embodiment of FIG. 3. The pumping mechanism 1 is reversibly connected to a housing 22 of the pump. A fluid conduit 21 of the pump 2 is provided above the pumping section 13 of the pumping mechanism 1. The eccentric ring 11 comprises a receptacle 111 for a motor shaft of a motor of the pump 2. In this way, an eccentric rotational movement of the ring 11 is converted via the pumping mechanism 1 into a movement of the pumping section 13. In particular, the multiplicity of locations 131 are subjected to a translational up and down movement due to a corresponding movement of the connecting elements 12. The pump 2 is, for example, a pump for administering medical fluid to a patient, e.g. an infusion pump.

FIG. 6a illustrates a method of operating a pump 2, in particular a pump for administering a medical fluid to a patient. The pump 2 comprises, in the present example, a pumping mechanism 1 according to the embodiment of FIG. 3. FIG. 6a shows the pumping mechanism 1, when the motor rotation angle 23 of the ring 11 is 0°. Due to the eccentric rotation of the ring 11, the first vertical connecting element 12a and the second vertical connecting element 12b are pulled downwards. This results in a downwards movement of the location 131a coupled via the pivot 122a to the first vertical connecting element 12a. The locations 131b and 131f coupled via the sleeper-bell-crank mechanism to the second vertical connecting element 12b are at the same time moved upwards. As a consequence, the diameter of the fluid conduit above locations 131b and 131f is reduced; less volume for the fluid is available in that area. Hence, a pinching zone 33 is created in the fluid conduit. As the motor proceeds its rotation, this pinching zone 33 moves.

This is illustrated for a motor rotation angle 23 of 90° in FIG. 6b. Here, no vertical force is exerted on the first and second vertical connecting elements 12a, 12b. Instead, the first and second intermediate connecting elements 12c, 12d are subject to a force exerted by the ring 11. The first and second intermediate connecting elements 12c, 12d are moved in a horizontal direction perpendicular to the vertical direction. Both, the first and the second intermediate connecting elements 12c, 12d are moved in the same direction. This movement results in a pulling movement on the second bell-crank 14d coupled to the second intermediate connecting element 12d. The same movement results in a pushing movement on the first bell-crank 14c coupled to the first intermediate connecting element 12c. As a consequence, the location 131d is pushed upwards, whereas the location 131c is pulled downwards. This results in the pinching zone 33 moving from the area of location 131f to the area of the location 131d.

As the motor rotation continues to a motor angle 23 of 180°, a vertical force is exerted on the first and second vertical connecting elements 12a, 12b, see FIG. 6c. The first and second vertical connecting elements 12a, 12b are pushed and pulled, respectively, upwards towards the pumping section 13. This results directly in an upwards movement of the location 131a to which the first vertical connecting element 12a is coupled. Due to the sleeper-bell-crank mechanism, this upward movement of the second vertical connecting element 12b results in a downwards movement of the corresponding locations 131b, 131f, this second vertical connecting element 12b is coupled to. Correspondingly, the pinching zone 33 progresses to the area above location 131a.

The cycle proceeds to a motor rotation angle 23 of 270°, depicted in FIG. 6d. A horizontal force in a direction opposite to the force exerted at motor rotation angle 23 of 180° is exerted on the first and second intermediate connecting elements 12c, 12d. The corresponding push on the second intermediate connecting element 12d is converted into a downwards movement of the corresponding location 131d. The pull on the first intermediate connecting element 12c is converted into an upwards movement of the corresponding location 131c. Thus, the pinching zone 33 progresses further along the fluid conduit 21 to the area above location 131c. The cycle completes at a rotation angle 23 of 360°, corresponding to FIG. 6a. In this way, the eccentric rotational movement of the ring 11 is converted in up-and downward movements traveling wavelike through the pumping section 13. Thus, a pinching zone 33 is provided which travels through the fluid conduit 21, hence generating a fluid flow in said conduit 21.

FIG. 7 shows a schematic illustration of another embodiment of the pumping mechanism 1. The depicted embodiment differs from the embodiment of FIG. 1 in that the sleeper-bell-crank mechanism is replaced by a double-bell-crank mechanism. This reduces the volume of the pumping mechanism 1. In the following, only the differences to the embodiment of FIG. 1 will be described. The second vertical connecting element 12b is coupled via a double-bell-crank mechanism to the pumping section 13 at a second location 131b and a third location 131f. The second and third location 131b, 131f are located at a first and a second end of the pumping section 13. The double-bell-crank mechanism comprises a first bell-crank 4a and a second bell-crank 4b. The first bell-crank 4a is coupled to a first linking element 41a which couples the second vertical connecting element 12b to the second location 131b of the pumping section 13. The second bell-crank 4b is coupled to a second linking element 41b which couples the second vertical connecting element 12b to the third location 131f of the pumping section 13. The first bell-crank 4a and the second bell-crank 4b are each coupled to fixation elements 20e, 20g for connecting the pumping mechanism 1 to a housing 22 of a pump 2. The second vertical connecting element 12b is coupled to the first and to the second bell-crank 4a, 4b via a pivot 42a, in particular a flexural pivot, and pivot 42b, in particular a flexural pivot, respectively. The first bell-crank 4a is coupled to the first linking element 41a via a pivot 411a, in particular a flexural pivot. The first linking element 41a is coupled to the second location 131b via the pivot 122b, in particular flexural pivot, at that location. The first bell-crank 4a is coupled to the fixation element 20e via a pivot 43a, in particular a flexural pivot. Correspondingly, the second bell-crank 4b is coupled to the second linking element 41b via a pivot 411b, in particular a flexural pivot. The second linking element 41b is coupled to the third location 131f via the pivot 122f, in particular flexural pivot, at that location. The second bell-crank 4b is coupled to the fixation element 20g via a pivot 43b, in particular a flexural pivot. In this way, a pumping mechanism 1 is provided with a reduced packing space as compared to the embodiment of FIGS. 1 and 3, for example.

FIG. 8 illustrates the pumping mechanism 1 according to another embodiment. The embodiment of FIG. 8 is a further specification of the embodiment of FIG. 7. Hence, the presently described embodiment has at least the features of the embodiment described with regard to FIG. 7. In this embodiment, packing space is further reduced by providing the fixation elements 20c, 20d, 20e and 20g in an opening of the respective bell-crank 14c, 14d, 4a and 4b they are coupled to. The coupling between the respective bell-crank 14c, 14d, 4a and 4b and its fixation element 20c, 20d, 20e and 20g is achieved through a pivot, in particular a flexural pivot, 142a, 142d, 43a and 43b, respectively. The last segment of the pumping section 13, the holding link 134, is coupled to a fixation element 20f. Linking elements 15c, 15d are provided that couple the first and second intermediate connecting elements 12c, 12d to the pumping section 13 at the respective locations 131c, 131d. In this specific embodiment, all pivots 122b, 122c, 122a, 122d, 122f, 121, 141a, 141d, 42a, 42b, 411a, 411b, 143c, 143d, 142c, 142d, 43a, 43b are provided as small-length flexural pivots, i.e. as bendable blades. In an alternative embodiment, the pivots 122c, 122a, 122d, 122f are provided as cross-axis flexural pivots.

FIG. 9 shows the pumping mechanism 1 according to an alternative embodiment. This embodiment differs from the embodiment of FIG. 7 in that no second vertical connecting element 12b is provided, but instead the first vertical connecting element 12a is coupled to the pumping section 13 via the double-crank mechanism. This further reduces the packing space of the pumping mechanism 1 considerably. The first and second intermediate coupling elements 12c, 12d are coupled to the pumping section 13 at locations 131c and 131d via a first and a second bell-crank 14c, 14d, respectively, in the manner described with regard to the embodiments of FIGS. 1 and 7. Linking elements 15c, 15d are provided between the respective first and second bell-crank 14c, 14d and the respective location 131c, 131d of the pumping section 13. All couplings are provided by pivots, in particular, flexural pivots. The first vertical connecting element 12a is coupled to the ring 11 via pivot 44 and to the pumping section 13 at a first location 131a via pivot 122a. Additionally, the first vertical connecting element 12a is coupled via a double-bell-crank mechanism to a second and third location 131b, 131f to the pumping section 13. The double-bell-crank mechanism comprises a first and a second bell-crank 4a, 4b. The first vertical connecting element 12a is coupled to the first and second bell-crank 4a via the pivot 44, in particular a flexural pivot. Hence, pivot 44 couples the first and second bell-crank 4a, 4b as well as the first vertical connecting element 12a and the ring 11 to each other. For example, the pivot 44 comprises two crossed bendable blades, wherein one of the bendable blades is a thinned out extension or section of the first vertical connecting element 12a and wherein the other of the bendable blades is a bendable blade extending in the horizontal direction between the first and second bell-crank 4a, 4b. The first bell-crank 4a is further coupled via pivot 411a, in particular a flexural pivot, to a first linking element 41a which in turn is coupled via the pivot 122b at location 131b to the pumping section 13. The coupling to the third location 131f is achieved through a coupling of the second bell-crank 4b via pivot 411b, in particular a flexural pivot, to a second linking element 41b which in turn is coupled to the third location 131f via the pivot 122f, in particular flexural pivot, at that location. The first and second bell-cranks 14c, 14d coupling the first and second intermediate connecting elements 12c, 12d to the pumping section 13 at locations 131c, 131d overlaps the first and second bell-cranks 4a, 4b of the double-bell-crank mechanism coupling the first vertical connecting element 12a to the pumping section 13 at the second and third location 131b, 131f. In this way, the space required by the pumping mechanism 1 is further reduced, allowing for a production of pumps 2 of smaller size. In one embodiment, the pumping mechanism 1 is produced as a single piece by 3D-printing. With 3D-printing it is possible to provide a pumping mechanism 1 with overlapping parts. This is not possible with traditional production methods as e.g. injection molding.

### LIST OF REFERENCE NUMERALS

1 Pumping mechanism
11 Ring
111 Receptacle for motor shaft
112 Eccentric applied to motor shaft
12 Connecting element
12a First vertical connecting element
12b Second vertical connecting element
12c First intermediate connecting element
12d Second intermediate connecting element
12e Further intermediate connecting element
121 Pivot between ring and connecting element
122 Pivot coupling connecting element to location
122a Pivot for coupling first vertical connecting element to location
122b (first) Pivot for coupling second vertical connecting element to location
122c Pivot for coupling first intermediate connecting element to location
122d Pivot for coupling second intermediate connecting element to location
122f (second) Pivot for coupling second vertical connecting element to location
13 Pumping section
131 Location on the pumping section
131a Center location
131b First end location
131c First intermediate location
131d Second intermediate location
131e Further locations
131f Second end location
132 Pumping element
132a-d Pumping element
133 Pivot coupling holding link to fixation element
134 Holding link
14 Bell crank
14c First bell crank (coupled to first intermediate connecting element)
14d Second bell crank (coupled to second intermediate coupling
141 Pivot coupling connecting element to bell crank
141c Pivot coupling first intermediate connecting element to bell crank
141d Pivot coupling second intermediate connecting element to bell crank
142 Pivot coupling bell crank to fixation element
142c Pivot coupling first bell crank to fixation element
142d Pivot coupling second bell crank to fixation element
143 Pivot coupling bell crank to link element
143c Pivot coupling first bell crank to link element
143d Pivot coupling second bell crank to link element
15 Linking element
15c First linking element
15d Second linking element
16 First bell crank of sleeper-bell-crank mechanism
161 Pivot coupling first bell crank and connecting element
162 Pivot coupling first bell crank and fixation element
163 Pivot coupling first bell crank and sleeper
17 Side bell-crank of sleeper-bell crank mechanism
17a First side bell-crank of sleeper-bell-crank mechanism
17b Second side bell-crank of sleeper-bell-crank mechanism
171 Pivot coupling side bell-crank and connecting element
171a Pivot coupling first side bell crank and connecting element
171b Pivot coupling second side bell crank and connecting element
172 Pivot between side bell crank and fixation element
172a Pivot between first side bell crank and fixation element
172b Pivot between second side bell crank and fixation element
18 Sleeper of sleeper-bell-crank mechanism
19 Linking element of sleeper-bell-crank mechanism
19a First linking element of sleeper-bell-crank mechanism
19b Second linking element of sleeper-bell-crank mechanism
20 Fixation element
20b-g Fixation element
2 Pump
21 Fluid conduit
22 Housing
23 Rotation angle of motor
3 Fluid volume
31 upstream incoming fluid
32 downstream outcoming fluid
33 Pinching zone
4a, 4b First and second bell-crank of the double-bell-crank mechanism
41a, 41b First and second linking element of the double-bell-crank mechanism
411a, 411b Pivot coupling the first and second bell-crank to the first and second linking element
42a, 42b Pivot coupling the first and second bell-crank to a vertical connecting element
43a, 43b Pivot coupling the first and second bell-crank to a fixation element
44 Pivot coupling the first vertical connecting element, the first and second bell-crank of the double-bell-crank mechanism and the ring

1. A pumping mechanism, in particular for a pump for administering medical fluid to patient, wherein:
the pumping mechanism is configured to act onto a fluid conduit of a pump, the pumping mechanism comprising a pumping section movable with respect to said fluid conduit such that a fluid flow through said fluid conduit is generated;
wherein the pumping mechanism comprises an eccentric ring for reception of a shaft of a motor of a pump and a multiplicity of connecting elements each operatively coupled to the pumping section at at least one of a multiplicity of different locations of the pumping section and operatively coupled to the eccentric ring in such a way that an eccentric rotational movement of the eccentric ring is converted into a translational movement of each of the multiplicity of different locations of the pumping section.

2. The pumping mechanism according to claim 1, wherein each connecting element is directly coupled to the eccentric ring via a pivot.

3. The pumping mechanism according to claim 1, wherein at each location of the multiplicity of different locations, a pivot is provided for flexibly coupling the respective connecting element to the pumping section.

4. The pumping mechanism according to claim 1, wherein the multiplicity of connecting elements face radially outward from the eccentric ring.

5. The pumping mechanism according to claim 1, wherein the multiplicity of connecting elements comprises a first vertical connecting element arranged on the side of the ring facing the pumping section, wherein the first vertical connecting element is directly coupled to the pumping section at a first location of the multiplicity of different locations via a pivot at the first location.

6. The pumping mechanism according to claim 5, wherein the multiplicity of connecting elements comprises a second vertical connecting element arranged on the side of the ring facing away from the pumping section, wherein the second vertical connecting element is coupled to the pumping section via a sleeper-bell-crank mechanism for redirecting the movement of the second vertical connecting element towards the pumping section.

7. The pumping mechanism according to claim 6, wherein the sleeper-bell-crank mechanism comprises a first bell-crank for coupling the second vertical connecting element to a sleeper and a first side bell-crank and a second side bell-crank for coupling the sleeper via a first linking element and a second linking element of the sleeper-bell-crank mechanism to the pumping section at a second location and at a third location of the multiplicity of different locations via a pivot at the second location and via a pivot at the third location, respectively.

8. The pumping mechanism according to claim 6, wherein the first and second vertical connecting elements are arranged along a vertical axis, wherein the vertical axis extends perpendicular to a plane defined by the pumping section at rest and wherein the vertical axis crosses a center of the eccentric ring.

9. The pumping mechanism according to one of the claim 1, wherein the multiplicity of connecting elements comprises at least one intermediate connecting element, wherein the intermediate connecting element is coupled to the pumping section via a bell-crank, and via a linking element, wherein the bell-crank is coupled to the pumping section at one location d) of the multiplicity of different locations via a pivot at that location.

10. The pumping mechanism according to claim 1, wherein the multiplicity of connecting elements consists of four connecting elements facing radially outward from the ring or wherein the multiplicity of connecting elements consists of eight connecting elements facing radially outward from the ring.

11. The pumping mechanism according to claim 1, further comprising at least one fixation element for connecting the pumping mechanism to a housing of a pump and/or wherein each of the multiplicity of connecting elements is a rod.

12. The pumping mechanism according to claim 1, wherein the pumping mechanism is formed as a single piece.

13. The pumping mechanism according to claim 1, wherein the pumping section comprises a multiplicity of pumping elements, wherein neighboring pumping elements of the multiplicity of pumping elements are pivotally coupled to each other at each of the multiplicity of different locations.

14. A pump, in particular for administering a medical fluid to a patient, wherein the pump comprises a fluid conduit, a motor, and a pumping mechanism according to claim 1.

15. A method of operating a pump, in particular a pump for administering a medical fluid to a patient, according to claim 1, the method comprising the steps of:
acting, through the pumping mechanism of the pump, onto the fluid conduit of the pump, wherein the pumping section of the pumping mechanism generates the fluid flow through said fluid conduit through a movement of the pumping section,
wherein the pumping section is moved by the translational movement of each of the multiplicity of different locations of the pumping section by the multiplicity of connecting elements which are operatively coupled to the pumping section at at least one of the multiplicity of different locations of the pumping section and wherein each of the multiplicity of connecting elements is coupled to the eccentric ring of the pumping mechanism for reception of the shaft of the motor of the pump, through conversion of the eccentric rotational movement of the ring into the translational movement of the multiplicity of different locations of the pumping section.

16. The pumping mechanism according to claim 2, wherein the pivot is a flexural pivot.

17. The pumping mechanism according to claim 3, wherein the pivot is a flexural pivot.

18. The pumping mechanism according to claim 4, wherein the multiplicity of connecting elements face radially outward from the eccentric ring at equal angular distances on the ring.
